(19) United States
(12) Patent Application Publication
(10) Pub. No.: US 2026/0024631 A1
(43) Pub. Date: Jan. 22, 2026

(54) AI NURSE PLATFORM: AN ARTIFICIAL INTELLIGENCE, CONVERSATIONAL (AI AGENTIC) AI NLP, GENERATIVE-AI & DEEP LEARNING LARGE LANGUAGE MODEL/S (LLM) DRIVEN VIRTUAL ASSISTANT/S FOR ENHANCED PATIENT PERSONALIZED CARE OUTCOMES & FACILITY EFFICIENCIES

(71) Applicant: Abijith Kariguddaiah, Dublin, CA (US)

(72) Inventor: Abijith Kariguddaiah, Dublin, CA (US)

(21) Appl. No.: 19/271,812

(22) Filed: Jul. 17, 2025

Related U.S. Application Data
(63) Continuation-in-part of application No. 18/777,782, filed on Jul. 19, 2024.

(51) Int. Cl. G16H 10/60; G16H 40/63

(52) U.S. Cl. CPC G16H 10/60; G16H 40/63

(57) ABSTRACT

A system and method for providing a modular virtual assistant platform powered by conversational AI, Machine Learning (ML), Natural Language Processing (NLP), Generative-AI (Gen-AI), and Large Language Models (LLMs), designed to improve patient-centred care and healthcare facility efficiency. The platform monitors vital health data, provides automated Q&A assessments, integrates with historical Electronic Medical Record (EMR) data for near real-time analysis and personalized recommendations. The platform addresses challenges such as staff shortages, patient care delays, and limited healthcare access. A modular orchestration framework is configured to coordinate multiple specialized AI agents, each continuously learning and designed to solve specific healthcare use cases with high reliability. These agents can be interconnected to collectively address complex clinical workflows. Key features include patient-nurse interaction, administrative task automation, data analysis and control, cloud-based infrastructure, device agnostic integration, and blockchain-enabled patient data ownership.

### CROSS-REFERENCE TO RELATED APPLICATION

This non-provisional application claims priority from a US Continuation in Part patent application Ser. No. 18/777,782 filed on Jul. 19, 2024, and hereby claims the benefit of the embodiments therein and of the filing date thereof.

### FIELD OF INVENTION

The present invention relates to the field of natural language processing (NLP), AI Agents, LLMs. and AI artificial intelligence (AI) in healthcare technology, and more specifically, to an AI-AGENT/AI/ML/NLP/GENAI/LLM-driven virtual assistant platform called AI Nurse that improves patient outcomes by providing automated Q&A assessments and monitoring vital health data, and combining with patients historical EMR (Electronic Medical Record) data, provides for near real time analysis and recommendations for the specific patient/event use case, and providing new efficiencies to all constituents including the patient and clinic/hospital/Pharma/Insurance/health-organization clinicians. The platform utilizes AI-Agents, Artificial intelligence (AI) and Machine Learning (ML) and Generative-AI (GEN-AI) algorithms with Deep Learning (DL) Large Language models (LLM) which constantly learns to deliver personalized and real-time conversational AI solutions and efficiencies to all constituents, especially the patients and clinicians, and in multiple languages and in their medium of choice (Email/SMS/Voice/WhatsApp/etc.).

### BACKGROUND OF THE INVENTION

In the field of healthcare, patient outcomes are heavily influenced by the quality and timeliness of patient-nurse assessments and care interventions, for example in ambulatory pre-operative preparations & post-surgical situations. A case can be made for many similar situations, e.g. in clinical trials, timely interventions (e.g., off hours) become necessary while patients are trying out new unproven drugs, and especially for patients in home situations. Similarly, for most of rural American health settings, where care services can be poor and even emergency care is unavailable for hard-to-reach situations. Hence, due to various factors such as limited healthcare resources, nurse & staff shortages, time constraints, insurance coverage constraints, and constantly increasing need for patient health interventions, patients often face challenges in accessing timely and personalized care. i.e., initial nurse assessments just to investigate the adverse event and may have to wait days to weeks/months and emergency care is cost prohibitive to most, potentially worsening their health issues. And this is all the more prevalent in rural healthcare, where access to care is always a challenge. And subsequent delays in discovery of the issues and successful interventions when it does arrive, leads to more complications and costs to the patient and entire health system. Clearly there is no near real time timely analysis of the patient's issue (collected data) for the same reasons and there is definitely no conjunction/investigation with the patient's historical data, which is never usually done due to the complexity and lack of bandwidth of the physicians to be able to access the required information with relevance and just in time when needed. Additionally, administrative tasks and the management of patient data, especially the accuracy of the data due to inherent human mistakes, can be cumbersome and time-consuming for healthcare providers, not to mention the costs. And in some instances, devastating to patient outcomes.

In the field of surgical optimizations and necessary cancellations, the lack of real-time visibility into critical pre-operative workups-such as external surgical clearances from specialists (including, but not limited to, cardiologists, diabetologists, and others), as well as internal financial clearances-hampers clinicians' ability to make timely, informed decisions. This lack of coordinated and transparent information flow often results in avoidable, costly surgical cancellations. Ultimately, the absence of timely and streamlined care coordination contributes to poorer patient outcomes, particularly in rural healthcare settings where access to resources is already limited and current solutions remain inadequate.

Existing solutions in the market offer limited capabilities in terms of AI-NLP based patient assessments, patient self-help queries, & data, facility staff queries and task executions using conversational AI approaches. Let alone combining it with historical data, to provide rendered analysis & situational recommendations. There exists a pressing need for both labor and cost efficiencies within healthcare delivery, particularly to reduce the burden on care providers by alleviating them from repetitive, low-value administrative tasks. Additionally, enhancing patient engagement through convenient, multilingual, and multi-modal communication channels-such as voice, SMS, or WhatsApp—is essential. Patients increasingly seek on-demand support to get their queries answered, verify assessment data within the EMR (Electronic Medical Record), reschedule appointments, request emergency interventions, or complete patient intake forms through AI-guided voice interactions. Further, conversational AI systems can streamline a wide range of patient engagement and clinic-level workflows. When integrated with wearable devices, these systems can enable real-time patient monitoring and personalized care interventions, ultimately improving efficiency and clinical outcomes. There is a need for an innovative platform that can leverage advanced AI-AGENT/AI/ML/NLP/GENAI/LLM algorithms, provide personalized assessments, personalized queries or task executions, personalized patient recommendations using deep learning LLMs, streamline administrative tasks conversationally using AI, alleviate nurse shortage issues, and seamlessly integrate with various healthcare systems, EMRs and wearable & non wearable devices for faster and in near real-time diagnosis and interventions with physician approvals, from a holistic perspective for superior patient outcomes. The capabilities of this platform is further enhanced by introducing an advanced agentic framework supporting a network of and specific specialized AI agents designed to address a wider range of healthcare facilities, health systems, ambulatory surgery center and provide tailored solutions for improved patient outcomes and healthcare efficiency.

### SUMMARY OF THE INVENTION

The proposed platform invention is an advanced Conversational AI-AGENT/AI/ML/NLP/GENAI/LLM-driven virtual assistant platform called AI Nurse that addresses the aforementioned challenges and aims to improve patient outcomes, and vastly improve surgery centers, ASC's (Ambulatory Surgical Centers), surgeon practices and other such health systems other than surgery, with health facility specific efficiencies. The platform provides custom automated Q&A assessments, patient-nurse engagement, patient & facility staff self-help conversational AI solutions, situational analysis & recommendation and monitoring of vital health data. It is device-agnostic and can integrate with various wearable devices and technology & health platforms through APIs.

Key features of the platform include patient-nurse engagement, conversational AI solutions for facility staff and nurses & patient use cases, administrative time savings, data analysis & control, custom AI/ML/NLP/GENAI/LLM algorithms, cloud-based infrastructure, clinic/hospital/Pharma/Insurance/health-organization specific use cases, personalized emergency help, modular and plug-and-play integration, wearable & non wearable integrations, and blockchain integration and AI-driven assessments, analysis and recommendations.

The AI Nurse platform will support both Public and private LLMs where the public LLM is SaaS based and provides holistic view of all information regarding said area such as Gastroenterology, or Anesthesia or Orthopedic Surgery, or vascular surgery, etc., to name a few. Private LLMs will be provided for the larger establishments to have a closed loop secure data/analysis and historic views of the patients exclusively, to support their data standards, and of the institution which manages it.

Furthermore, the AI platform is augmented with an advanced AI agentic framework, comprising a plurality of specialized AI agents. Each AI agent is configured to solve specific use cases for a healthcare facility, staff, or patient. These AI agents are designed to perform particular tasks with high reliability while minimizing or eliminating hallucinations, thereby ensuring highly reliable outputs.

The platform further introduces a neural network of AI agents, or an agentic framework, which enables these specific AI agents to be chained or interconnected to collectively solve more complex problems for a facility, physicians, or nursing staff-all automatically and in near real-time, making it optimal and seamless for the user.

In addition to the key features, the platform offers additional features such as patient data analysis & control, AR/VR (Augmented Reality/Virtual reality) device integration and data analysis, adverse event monitoring, marketplace for assessments, AI AGENT/AI/ML/NLP/GENAI/LLM-powered recommendations, monitoring and early diagnosis, and integration with AI platforms including Meta's Llama, Google's Gemini, Open Ais GPT-4 & Amazons Lex, and any other open AI platforms.

These features work together to provide a comprehensive solution that improves patient outcomes, enhances efficiency, and facilitates personalized care in healthcare settings. The platform leverages the power of AI AGENT/AI/ML/NLP/GENAI/LLM and integrations with wearable & non-wearable technology to enable collection of situational data, preventive care, early diagnosis, and just-in-time care interventions.

### BRIEF DESCRIPTION OF THE DRAWINGS

FIG. 1 is a schematic representation of the AI Nurse Assessment, analysis & recommendation platform architecture with the various modular components according to an embodiment of the invention.

FIG. 2 is a flowchart illustrating the process of patient-nurse engagement and assessment generation using AI-NLP (Natural Language Processing) according to an embodiment of the invention.

FIG. 3 is a flowchart depicting the deep Learning LLM (Large Language Model) business process of both public and private LLMs for surgeries in this instant example according to an embodiment of the invention.

FIG. 4 is a flowchart illustrating the further inner workings of a typical LLM (Large Language Model) and analysis process according to an embodiment of the invention.

FIG. 5 is a flowchart demonstrating the Generative AI-driven rich content process flow according to an embodiment of the invention.

FIG. 6 is a flowchart architecture of an AI-powered web application framework according to an embodiment of the invention.

FIG. 7 is an exemplary flowchart of a networked system of modular artificial intelligence (AI) agent collaboratively managing patient workflows according to an embodiment of the invention.

FIG. 8 is an embodiment of a modular AI agent workflow.

FIG. 9 is an AI agentic architecture comprising a multi-layered cognitive framework structured into three primary modules according to an embodiment of the invention.

### DETAILED DESCRIPTION OF THE INVENTION

The following detailed description of the invention provides a more thorough understanding of the various features and functionalities of the AI Nurse Assessment, analysis & recommendation platform. It should be noted that the invention is not limited to the specific embodiments described herein but can be implemented in various other forms for various types of healthcare organizational patient engagement use cases without departing from the scope of the invention.

In one embodiment, the AI Nurse platform is augmented with an advanced AI agentic framework, comprising plurality of specialized AI agents. Each agent is configured to solve specific use cases for a healthcare facility, staff, or patient. These AI agents are designed to perform particular tasks with high reliability and are characterized by their ability to learn continuously while minimizing or eliminating hallucinations, thereby ensuring highly reliable outputs.

In one exemplary embodiment, the platform further introduces a neural network of AI agents, or an agentic framework, to the present architecture, comprising a neural network of interoperable artificial intelligence (AI) agents, each designed to perform a distinct task in broader clinical or administrative workflow. These specific AI agents can be chained or interconnected in real time to collectively execute more complex problems for a facility, physicians, or nursing staff that would generally require human environment. This neural network configuration allows for the creation of comprehensive AI solutions by combing the functionalities of individual, task-specific agents.

Each AI agent within this framework operates semi-autonomously and can expose standardized input/output protocols allowing seamless data handoff and instruction flow between agents. A multi-segment AI sequence may be employed during a patient intake process. For instance, a single complex AI solution can be formed by combining two or more distinct AI agents. As an example, a patient intake process can be managed by a first AI agent, such as a Patient Intake Agent, which guides the patient through from completion using conversational AI, asking questions, and transcribing answers. Is agent may leverage natural language processing (NLP), optical character recognition (OCR), and structured document synthesis techniques to generate a structured dataset from unstructured responses. Upon completion, this first agent then seamlessly hands over the collected data to a second AI agent, such as an EHR AI Agent, responsible for validating, enriching, and updating records of the patient data within an Electronic Health Record (EHR) system. The EHR AI Agent can correct any detected mistakes and update the EHR with the new patient entry. If inconsistencies are detected, this agent may route the data to a third agent, which triggers an approval request to a staff member or supervisor before EHR submission.

The platform may further employ a dynamic agent planner, which adapts the agent sequence based on patient conditions, facility policies, or environment parameters. For instance, if a patient is identified as high-risk, the intake chain may automatically include additional screening agents or notify human staff.

In another embodiment, the AI Nurse platform's framework provides for a comprehensive network of AI agents specifically tailored for tasks associated with patient engagement including but not limited within a hospital system, surgery center, and/or an ambulatory surgery centre. It is further configured with a graphical workflow design interface, enabling clinicians, administrators, or IT staff to design, modify, and deploy custom AI-driven workflows without writing code. Through a drag-and-drop visual editor, users can select from a library of pre-trained AI agents—each performing atomic tasks such as patient communication, form parsing, symptom screening, EHR data synchronization, or staff alerting—can connect them in logical sequences or parallel branches. This interface enables the “daisy chaining” of AI agents to solve complex clinical or administrative problem statements specific to the user's context. For instance, a clinician can build a workflow where a first agent collects preliminary complaints, followed by a second agent that categorizes urgency, then a third agent that coordinates available slots based on urgency level and clinician availability.

In another embodiment, the platform includes a modular FAQ Automation Agent configured to ingest and respond to frequently asked questions across a variety of domains. While examples in healthcare may include dental procedures, surgical preparations, or specialty medication protocols, the agent is not restricted to clinical environments. This AI agent is designed to function across any industry-such as finance, insurance, education, or enterprise operations—by leveraging domain-specific libraries of structured or unstructured content. These may include internal policy manuals, product documentation, customer service transcripts, or learning management systems. The AI-driven FAQ Agent uses LLM-powered semantic search and retrieval-augmented generation (RAG) to respond contextually and accurately, with low hallucination risk, and can be configured to continuously improve based on interaction feedback. Deployment options include internal staff enablement, customer-facing chatbots, and cross-department training systems. The agent's modularity allows it to integrate seamlessly with pre-existing enterprise knowledge bases or EMR systems, thereby enabling automation of query resolution, training dissemination, and documentation support in real time.

### Implementation of AI Agentic Framework

In a specific embodiment, the AI Nurse platform, through its AI agents and framework, addresses the prevalent issue of surgery cancellation and the inability to find timely replacements, a problem impacting approximately 300 million surgeries globally each year. Thus, the platform overcomes these limitations by utilizing AI, AI-NLP, and dedicated AI agents to provide real-time, 360-degree visibility into each patient's workup activity. Specialized AI agents facilitate easy scheduling and perform “4th party assessments” to gather comprehensive patent information. In instances where a patient is not cleared in time, the system, via its AI agents, can identify and garner approval from a timely replacement patient, thereby ensuring that the scheduled surgery appointment and facility resources are not wasted or cancelled. All relevant patient scenario information is made available at the clinician's fingertips through conversational AI agents, leading to significant efficiencies, cost savings, and nursing efficiencies for the facility and clinicians. The AI agent is further configured to engage in conversational interactions to respond to queries related to a patient's procedure and overall care journey, and to autonomously initiate or perform appropriate actions based on those interactions, all in near real-time.

In one of the embodiments, the AI nurse and its AI agent's platform framework enhance post-surgery care, particularly in wound examination and analysis, which is traditionally performed manually requiring patient clinic visits. Here, the platform captures the image and video data of the patient's wound, which is then provided to the care team for analysis. Furthermore, the AI Nurse platform, through its AI agents, performs healing progression analysis against current industry standards, such as PWAT (Pressure Ulcer Scale for Healing) wound healing standards. This provides clinicians with AI-driven analytical support to make more accurate determinations, leading to better patient outcomes, especially in fields like but not limited to plastic surgery and orthopaedic surgery. Furthermore, this AI agent is further configured to engage in conversational interactions to respond to queries related to a patient's procedure and overall care journey, and to autonomously initiate or perform appropriate actions based on those interactions, all in near real-time.

In another embodiment, the AI Nurse platform is configured to support real-time or near real time mental health assessments, with the objective of detecting, triaging, proactively intervening in cases of psychological distress or potential self-harm. Recognizing the widespread and growing prevalence of mental health challenges across patient populations, this embodiment focuses on timely identification of mental health indicators using AI-driven, evidence-based screening methodologies. The platform may include a specialized agent or a set of collaborating agents, which is capable of conducting evaluations aligned with recognized diagnostic frameworks, including but not limited to the DSM-5 (Diagnostic and Statistical Manual of Mental Disorders, Fifth Edition) assessment measures, as published by the American Psychiatric Association. The real time nature of this agentic evaluation allows for early detection of acute episodes, such as suicidal ideation, aggression, or severe anxiety.

In one specific embodiment, the AI Nurse platform functions as a voice-enabled, patient-facing engagement layer that seamlessly integrates with third-party Electronic Health Records (EHR) systems, such as EPIC®, Cerner®, or Allscripts®, among others. This platform, through one or more AI agents, serves as a front-end extension to these EHR systems. These agents enable patients to interact with their personal health records, clinical instruction, and care pathways using natural language speech or text interfaces. This embodiment further comprises of a Voice Interface Module, capable of real-time transcription, intent recognition, and dialogue management; a Patient Interaction engine, which interprets user inputs, maintains session contact, and invokes appropriate AI agents for further actions-such as retrieving lab results, explanation of medication regimes, etc.; and a Secure EHR Connector Layer, responsible for mapping AI-generated requests to EHR API calls and securely retrieves data.

In a further embodiment, the AI Nurse platform includes a specialized AI Nurse PDF Agent designed to streamline the traditionally time-consuming process of collecting complex historical patient information during the intake phase. This agent uses conversational AI to interact directly with the patient in a natural, dialogue-based format, simplifying the data gathering experience, and completing the patient intake form in a fluid, easy manner. Simultaneously, the agent can address any clarifications or questions raised by the patient, verify the accuracy of the collected information upon completion, and obtain a verbal and, where required, written sign-off from the patient. The system further supports integration with existing patient intake systems utilized by healthcare facilities, enabling seamless incorporation of the completed and verified data into standard clinical workflows. The agent then provides the patient with the completed PDF documents for final approval before it is committed to the physician's records, offering a user-friendly and efficient patient intake experience. This in-turn reduces clerical errors, increasing both clinical reliability and regulatory defensibility. The PDF agent may also built-in logic for flagging incomplete sections, reconciling ambiguous responses, or escalating to human staff for manual review if inconsistencies are detected. These completed documents can be digitally signed and stored in a structured repository linked it the patient's EHR profile.

Furthermore, the AI Nurse platform supports integration with and functionality across various AI-NLP front ends, including but not limited to Alexa, Google Home, and Siri. This ensured broad accessibility and convenience for patient engagement through their preferred voice-activated devices. Through these integration, patients can access AI Nurse capabilities, enabling voice-driven interaction for healthcare tasks such as but not limited to medication reminders, appointment scheduling, symptom reporting, retrieval of lab results, pre & post-operative instructions, etc.

In a novel embodiment, the AI Nurse platform provides infrastructure that allows patients to store, tokenize, and monetize their personal health data using block chain technology. Each patient's data-including structured EHR data, AI-driven assessments, genetic information, and tracking, may be stored and hashed onto a permission or public block chain, optionally by use case. A data token (Non-Fungible Token (NFT) or fungible data coin) is generated and associated with the patient's digital wallet.

In yet another exemplary embodiment, the smart platform may be configured to operate as a pseudo primary care physician or digital longitudinal caregiver under the full ownership and control of the patient for a specific prescribed fee. This AI agent, referred to as the AI Caregiver Agent, is designed to maintain a persistent and evolving knowledge base of the patient's health history and serve as an intelligent, proactive healthcare industry. This AI Caregiver Agent would perform personalized healthcare management including but not limited to, physical and mental health monitoring, chronic disease management and care plan optimization, automated scheduling and reminders for screening and vaccination, health behaviour coaching, regular updating care strategies, etc. This AI Caregiver Agent, may interface with the patient's EHR records, wearable devices, remote monitoring tools, and historical clinical documentation to continuously refine its personalized care model. It is capable to generating long term health reports, risk predictions, and wellness trends, which can be shared by human providers upon request.

In an embodiment, the AI Nurse platform and its agents facilitate proactive medication adherence and pharmacy driven patient engagement to support optimal therapeutic outcomes. This embodiment includes one or more AI Agents, which coordinate between parties, prescribers, and pharmacies, to monitor prescription dosage schedules, automatically send timely refill reminders or initiate refill requests with pharmacies, check for missed doses or non-adherence patterns using inputs from smart pillboxes, engage in real-time conversations with patients to assess tolerance, side effects, and symptom improvements, etc. The system may also generate personalized medication instructions, adherence dashboards visible to providers or caregivers, automated alerts fro drugs requiring close monitoring, etc.

In an embodiment focused on dynamic symptom control, the AI agent's framework will provide near real time pain management assessment capabilities using one or more dedicated AI agents. Leveraging established pain assessment methodologies such as Norcross Pain Level Scale, the platform can gauge a patient's current pain intensity, monitor pain progression trends over time, correlate pain symptoms with existing treatment plans, trigger adaptive medication workflow with doctor's approval, etc.

In one variation of dynamic symptom control, the platform AI agentive framework has been set up to support nurses, physicians and clinical staff to see the patient's path through the system undertaking a 360-degree view from intake to post care. By leveraging the real-time conversational AI capabilities of the platform clinical users can access and act on an important aspect of the information in real time. Also, the AI platform allows the implementation of corrective actions through a structures command interface dedicated to a specific Surg360 AI agent to facilitate various items, including, but not limited to, the cancellation of surgical procedures and notifications to stakeholders, and modifying the patient's Electronic Medical Record (EMR) with the clinical information obtained.

In yet another embodiment, the AI agentic framework is designed to provide patients with context-based education resources specifically approved by the health institution. This could include individualized information such as dietary restrictions, prescribed medications, preparation/recovery from a procedure, exercises to follow, and any other educational resources. The AI ensures that the patient received timely, contextual, accurate, and approved information that promotes and influences compliance and engagement during their care experience.

In a different embodiment, the AI agentic framework enables intelligent collaboration among patients, physicians, registered nurses (RNs), and other clinical personnel through an AI Triage Assist Agent. The AI agent is available on demand and ingests and evaluates information to assist triage conversations with patients, delivering information relevant to the timely and substantive discussions with care teams, facilitating access to clinical data relevant to a specific patient if necessary, patient history, and situational information and insights pertinent to the healthcare facility, thereby assisting the team in making smarter and coordinated decisions.

In a further embodiment, the AI Nurse platform-specifically the AI agentic framework—was designed to function as an isolated, private AI solution for a clinic or healthcare entity. Each entity may use their own private Large Language Models (LLM), allowing signals from the clinic-specific use cases, workflows, patient histories, and unique patient interactions. Together these deployments will be designed to provide the most local, individual, and customized outcome-based care claims-specific to deeply understand the needs of the clinic, staffing, and location demographics.

In an additional embodiment, the AI Nurse platform permits the collection and anonymous extraction of private deployment use case data with the purpose of creating domain based public LLMs from a portfolio of customers. These public LLMs can be organized around specialties like face-lifts, Gastroenterology, or orthopaedic surgery (e.g., a 60-year old diabetic with cardiovascular comorbidity who is undergoing a colonoscopy). institutions will have access to these public models in order to build out their company AI Nurse platform. Access to domain-based public LLMs may be available for purchase through a Software-as-a-Service (SaaS) subscription.

The various embodiments of the AI Nurse platform and its AI agent's framework are specifically designed to provide significant value for rural health settings and veterans administration. In these settings, the AI Nurse framework provides voice activated self-assessments to eliminate travel needs, remote AI agent consultations when clinicians are unavailable, workflow automation for rural staff, veteran tailored questionnaires, etc. to promote scalable, equitable care delivery to populations often overlooked by conventional systems.

In an embodiment for a more comprehensive patient experience, the AI Nurse and its AI agent's platform framework allow for the purchase of physical products through integrated e-commerce platforms such as but not limited to Amazon, institutional dispensaries, or private retail websites. This functionality can be triggered as part for a voice assessment engagement with the patient, for example, recommending and facilitating the purchase of scar healing cream for wound care.

The AI Nurse and AI agent's platform framework will enable healthcare facilities to streamline financial interactions. This includes allowing patients to ask questions about their billing statements, coverage clarification, co-payment breakdowns, pending insurance reimbursements, balance due timelines, and other financial inquiries through a conversational AI agent. This AI agent is capable of interpreting medical billing codes, retrieving context from back end billing systems, and responding in layperson-friendly language.

In a further embodiment, the AI Nurse platform supports intra-facility coordination, enabling surgery nurses or care coordinators to proactively engage finance teams when preparing for scheduled packages, inquire about patient's insurance authorization, confirm payment guarantees, identifying billing flags, trigger escalation if financial clearance is pending, etc. Through the conversational AI agent, surgery nurses can ask finance team questions to resolve such issues, thereby helping to ensure that surgeries are not cancelled due to administrative or financial reasons.

In all embodiments of the invention, the AI agent is further configured to engage in natural, conversational interactions to answer any patient queries related to their procedure and overall care journey. These queries may include but not limiting to clarifications regarding pre-operative or post-operative steps, ongoing treatment plans, medication regimens, or logistical aspects such as appointment scheduling or billing. Upon receiving and understanding the query, the AI agent is capable of autonomously initiating appropriate actions, including but not limited to data retrieval, updates to the EMR system, form population, task escalation, or real-time staff alerts. All such functions are performed automatically and in near real-time, thereby delivering a seamless, responsive, and patient-friendly experience across the care continuum.

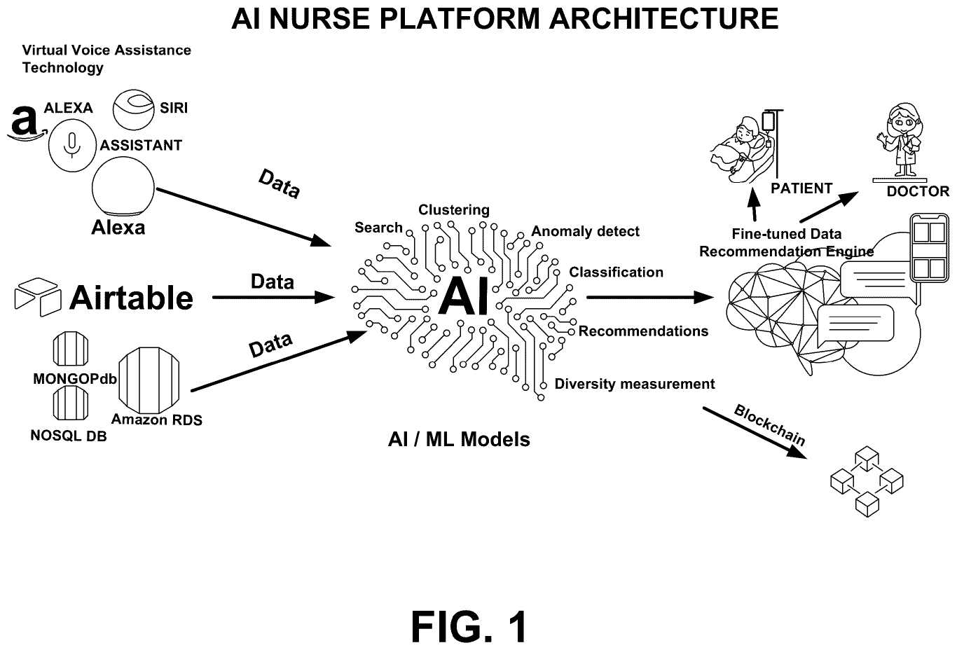

FIG. 1 illustrates the architecture of the AI Nurse Assessment, analysis & recommendation platform. The platform operates on a cloud-based infrastructure, which enables scalability, real-time data processing, and accessibility from multiple platform systems, and devices. The core component of the platform is an AI AGENT/AI/ML/NLP/GENAI/LLM based algorithm/s that continuously learns and improves based on patient data and feedback, eliminating the need for direct contact between patients and nurses/staff for inquiries. And applicable to many use cases where the need for near real-time patient-engagement becomes a necessity for better outcomes.

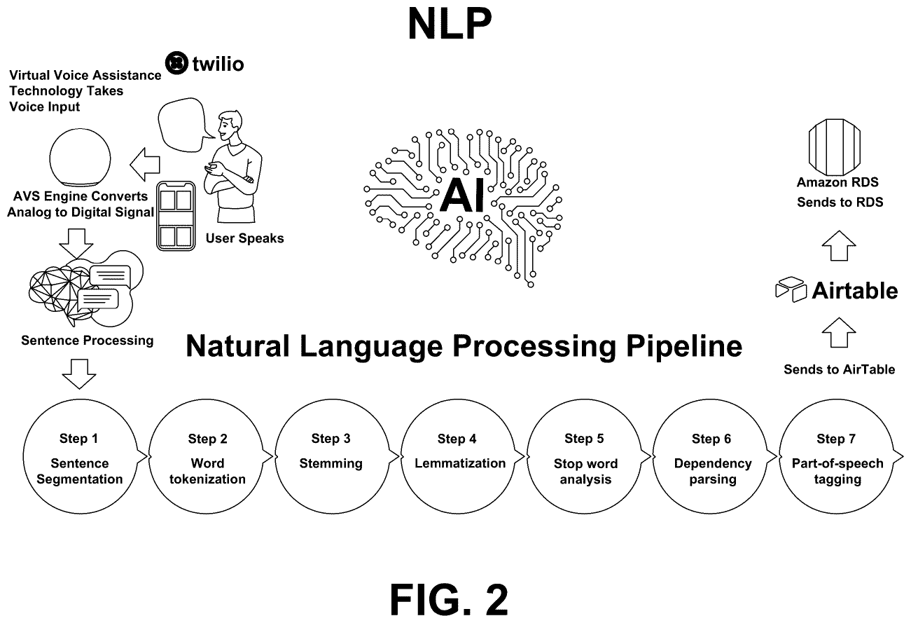

FIG. 2 presents a flowchart depicting the process of patient-nurse engagement and assessment generation. Patients can engage with nurses through choice of voice supported platforms, wearable & non-wearable devices for various clinic/hospital/Pharma/Insurance/health-organization related interactions, including scheduling, pre/post procedure compliance, emergency, patient self-help and vitals checks & analysis (i.e. an instant on-demand health check analysis, where patient gets a status on their current health based on all available data for personalized health).

The platform utilizes natural language processing (NLP) techniques to interact & understand patient queries, generate custom appropriate assessments (e.g. based on underlying conditions), captures and provide responses to care team, and clinic approved recommendations to patients. Platform utilizes Custom AI/ML/NLP/GENAI/LLM algorithms from providers like OpenAI (GPT-4), Amazon Lex, Microsoft, IBM, and Google AI and incorporated to enhance patient outcomes and automate Q&A assessments, analysis & recommendations.

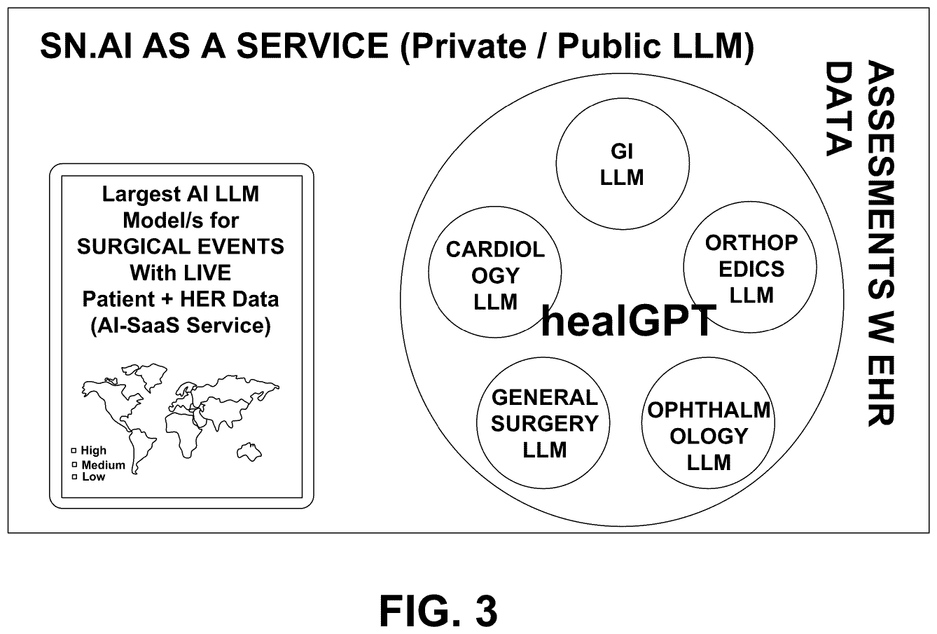

FIG. 3 depicts AI Nurse platform's deep learnings LLM offerings which include private and Public LLMs. The deep learning LLMs cater to their constituents by constantly learning and being able to answer more and more sophisticated queries and is directly proportional to time and quality data harnessing & consumption. The main difference being 1. Private Ilms are catering to the health organization and its patients and internal workforce, while the public LLMs a generic representation of the models inherent to the specific use case (e.g. Wound care in Gastroenterology, or wound care in orthopedic surgeries, etc.) and caters to the consuming public via a public SaaS transaction-based approach.

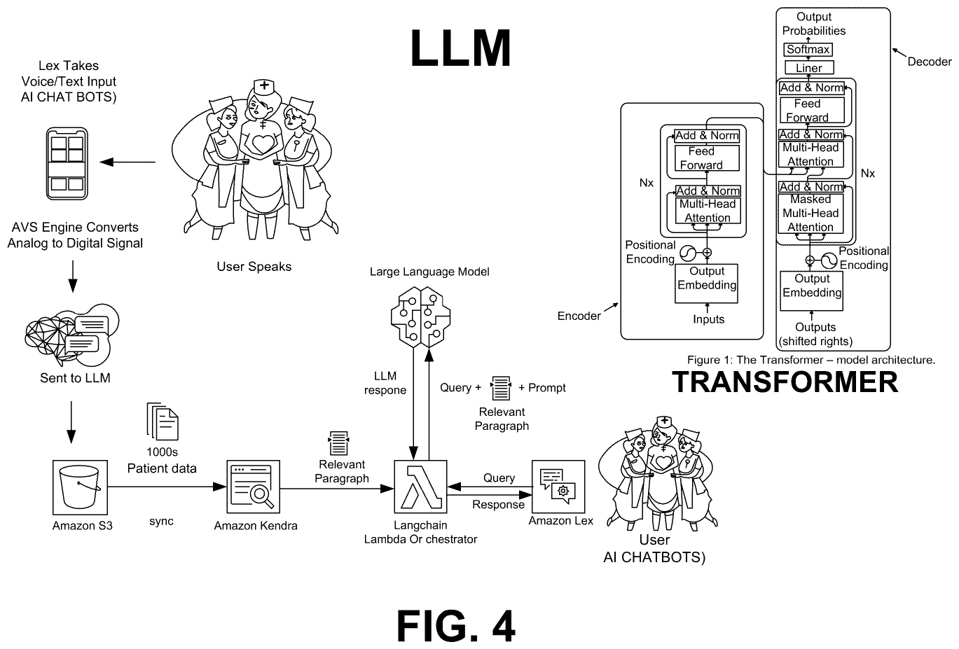

FIG. 4 illustrates the specialized LLM-Heal GPT's Algorithmic flow of data collection and threshold pointer to access & train the AI. The Heal GPT Algorithm for Personalized Healthcare Recommendations comprises a series of steps and components.

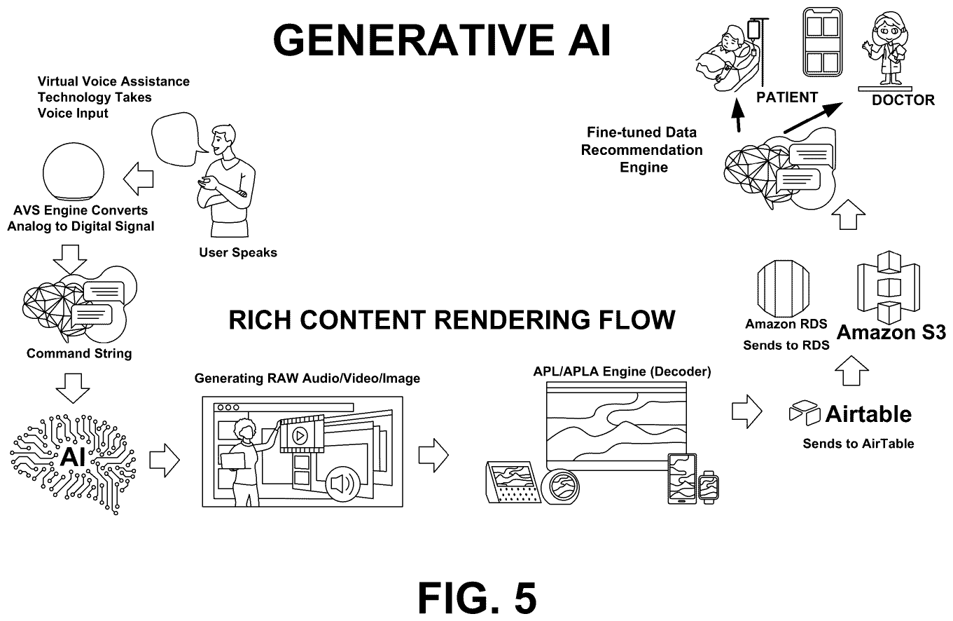

FIG. 5 illustrates the rich content rendering workflows; the ability for the AI engine to render custom contextualized rich audio and video content to benefit the clinics and the patients, so that they are better educated, become easily compliant and have a superior patient satisfaction & experience.

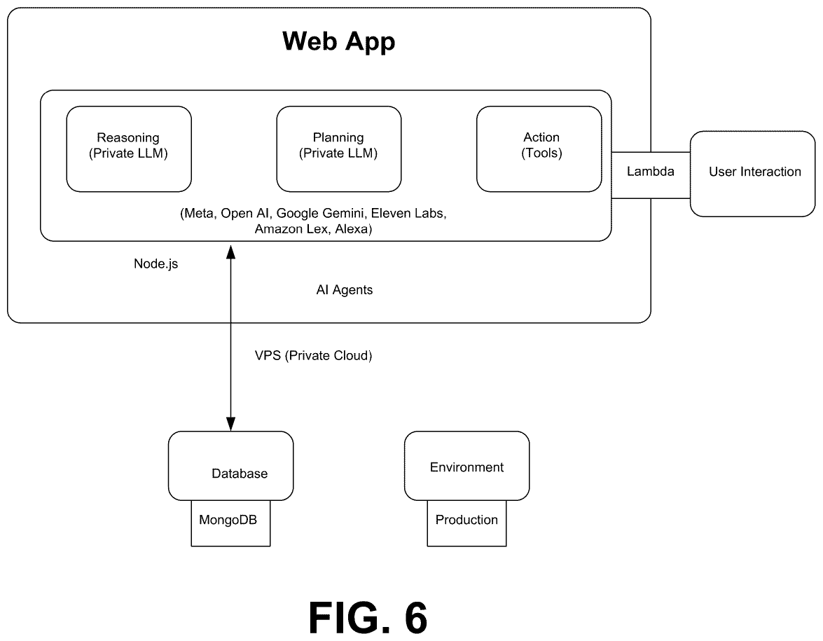

FIG. 6 illustrates a modular AI-powered web application architecture deployed in a private cloud environment, comprising three core agent modules-Reasoning, Planning, and Action—each powered by private Large Language Models (LLMs). The Reasoning module interprets user inputs, the Planning module generates structured execution plans, and the Action module interfaces with tools and APIs to perform tasks. These modules are orchestrated by a Node.js backend that manages inter-agent communication, connects to a MongoDB database for persistent storage, and integrates with serverless Lambda functions to relay outputs to user-facing interfaces such as chatbots or dashboards. The system supports integration with external AI providers (e.g., OpenAI, Meta, Google Gemini) for fallback or specialized tasks, and is designed for horizontal scalability, secure deployment, and real-time responsiveness in production environments.

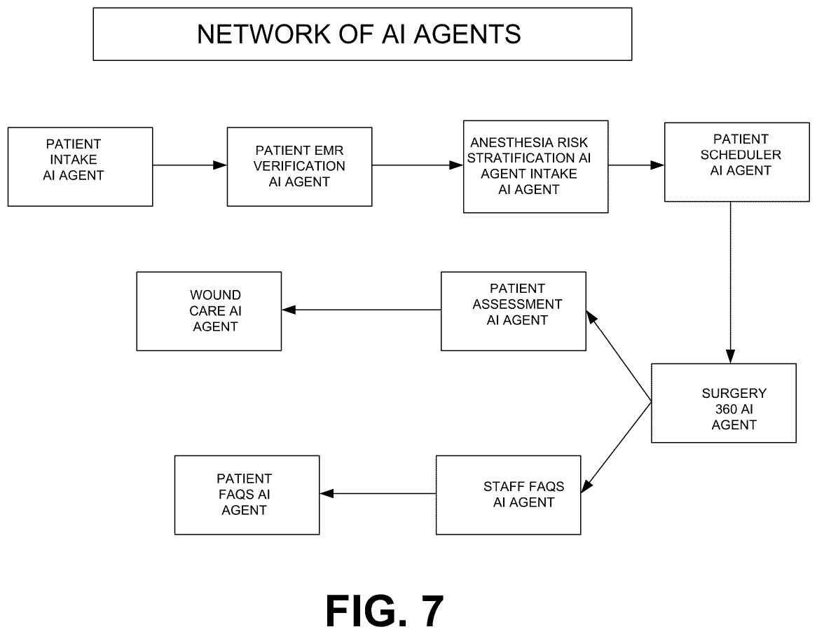

FIG. 7 depicts a modular network of domain-specific AI agents designed to collaboratively manage various stages of patient care, from intake to surgical planning and post-operative support. The workflow begins with a Patient Intake AI agent capturing preliminary data, which is verified by a Patient EMR Verification AI Agent and assessed for anaesthesia risk. A Patient Scheduler AI Agent then coordinates surgery logistics, feeding data into a central Surgery 360 AI Agent that consolidates insights from a Patient Assessments AI Agent. This assessment data further informs specialized agents like the Wound Care AI Agent for postoperative planning, the Staff FAQs AI Agent for generating internal clinical guidance, and the Patient FAQs AI Agent for translating medical content into patient-friendly communication. Directed arrows indicate data flow and task transitions between agents, enabling asynchronous, modular operation across a scalable, API-based infrastructure.

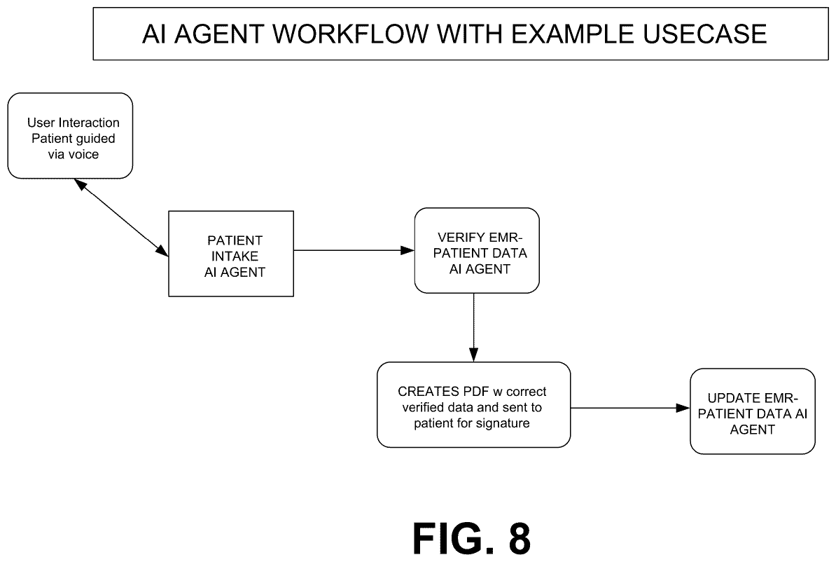

FIG. 8 illustrates a modular AI agent workflow designed to automate patient intake, verification, and EMR updating through a voice-guided interface and intelligent document handling. The process begins with a Patient Intake AI Agent that captures and structures patient data via natural language interaction. This data is then validated by a Verify EMR-Patient Data AI Agent, which compares it against existing medical records. Once verified, a PDF is automatically generated and sent to the patient for digital signature. Upon receipt, the signed and validated information is processed by an Update EMR-Patient Data AI Agent, which integrates the data into the electronic medical record in compliance with healthcare standards. The entire workflow is modular, supports real-time operation, and may be enhanced with biometric authentication and staff notifications for secure, efficient clinical documentation.

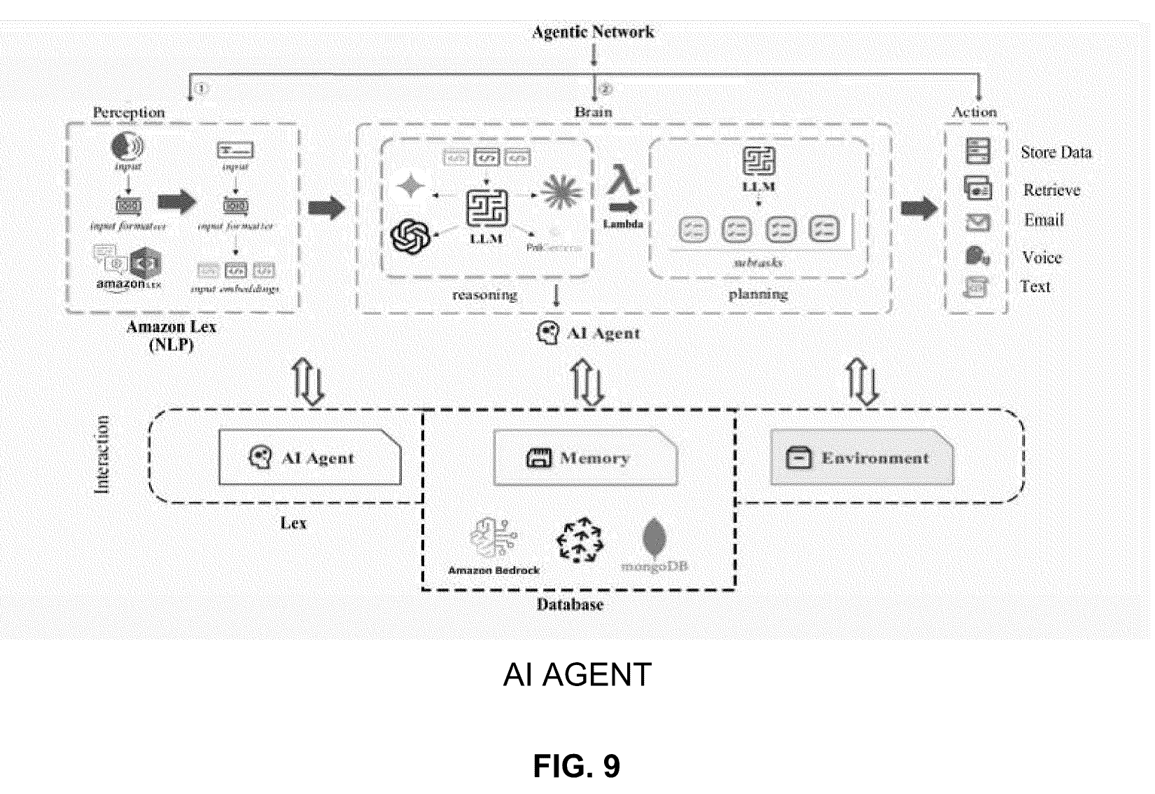

FIG. 9 is an AI agent architecture comprising a multi-layered cognitive framework structured into three primary modules: Perception, Brain, and Action, integrated within an agentic network and database-backed environment. The Perception layer utilizes natural language processing (NLP) capabilities, including Amazon Lex and other NLP engines, to ingest and interpret multimodal user inputs (text, speech, gestures) into structured intent representations. These intents are transmitted to the Brain layer, which includes two primary submodules-Reasoning and Planning—each powered by private or third-party large language models (LLMs), including but not limited to OpenAI, Hugging Face, or Amazon Bedrock. The Reasoning module processes context using logic engines, vector search tools, and APIs to understand user needs and system context, while the Planning module constructs a sequence of actions and decision paths based on contextual goals. Serverless function triggers (e.g., AWS Lambda) coordinate inter-agent task distribution and external tool invocation. The output of the planning process is executed by the Action layer, which handles downstream operations such as data storage, retrieval, email dispatch, voice output, and messaging through appropriate service endpoints. At the core of this architecture, a unified AI Agent interfaces with three internal components: an Interaction Handler (Lex), a Memory module (backed by Amazon Bedrock and MongoDB for persistent and contextual memory), and a programmable Environment module that reflects the agent's current execution and operational scope. Bidirectional communication pathways allow the AI Agent to iteratively access memory, interpret instructions, and adjust to dynamic changes in user input or system environment, thereby enabling continuous adaptive learning and task optimization.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A conversational artificial intelligence (AI) platform for enhanced patient personalized care outcomes and facility efficiencies, the platform comprising:
a cloud-based infrastructure configured for scalability and real-time data processing;
one or more Large Language Models (LLMs), comprising:
at least one public LLM providing a holistic view of information for specific medical domains; and
at least one private LLM for closed-loop, secure data analysis and historical views of patients specific to an institution;
an AI agentic framework comprising a plurality of specialized AI agents, wherein each AI agent is configured to perform a distinct task within a clinical or administrative workflow and learn continuously;
a patient-nurse engagement module configured to provide custom automated Q&A assessments and conversational AI solutions;
a data integration module configured to:
monitor vital health data;
integrate with historical Electronic Medical Record (EMR) data; and
Integrate with wearable and non-wearable devices and technology platforms via Application Programming Interfaces (APIs);
an analysis and recommendation engine configured to provide near real-time analysis and personalized recommendations based on collected data and historical EMR data;
a communication module configured to deliver conversational AI solutions in multiple languages and across various mediums including email, SMS, voice, and WhatsApp; and
a control circuit configured to interlink said plurality of specialized AI agents to collectively solve complex problems automatically and in near real-time.

2. The platform of claim 1, wherein the AI agentic framework further comprises a neural network of interoperable AI agents, each operating semi-autonomously and exposing standardized input/output protocols for seamless data handoff and instruction flow.

3. The platform of claim 1, wherein one of the pluralities of specialized AI agents is a Patient Intake Agent configured to guide a patient through intake completion using conversational AI, ask questions, transcribe answers, and leverage NLP, Optical Character Recognition (OCR), and structured document synthesis techniques to generate a structured dataset.

4. The platform of claim 3, further comprising an EHR AI Agent configured to receive collected data from the Patient Intake Agent, validate, enrich, and update records of the patient data within an Electronic Health Record (EHR) system.

5. The platform of claim 1, further comprising a dynamic agent planner configured to adapt the agent sequence based on patient conditions, facility policies, or environment parameters.

6. The platform of claim 1, wherein one of the pluralities of specialized AI agents is configured to provide real-time, 360-degree visibility into a patient's pre-operative workup activity and to identify and gain approval from a replacement patient in case of non-clearance, to prevent surgical cancellations.

7. The platform of claim 1, wherein one of the pluralities of specialized AI agents is configured to capture image and video data of a patient's wound, provide it to a care team for analysis, and perform healing progression analysis against industry standards.

8. The platform of claim 1, wherein one of the pluralities of specialized AI agents is configured to perform real-time or near real-time mental health assessments, including detecting, triaging, and proactively intervening in cases of psychological distress or potential self-harm, utilizing evidence-based screening methodologies like DSM-5 assessment measures.

9. The platform of claim 1, configured to function as a voice-enabled, patient-facing engagement layer that seamlessly integrates with third-party Electronic Health Record (EHR) systems, further comprising:
a voice interface module capable of real-time transcription, intent recognition, and dialogue management;
a patient interaction engine configured to interpret user inputs, maintain session context, and invoke appropriate AI agents; and
a secure EHR Connector Layer responsible for mapping AI-generated requests to EHR API calls and securely retrieving data.

10. The platform of claim 1, further comprising a specialized AI Nurse PDF Agent designed to streamline collection of historical patient information during intake using conversational AI, interact directly with the patient, verify accuracy, obtain sign-off, and integrate with existing patient intake systems.

11. The platform of claim 1, further comprising an infrastructure that allows patients to store, tokenize, and monetize their personal health data using blockchain technology, wherein a data token is generated and associated with the patient's digital wallet.

12. The platform of claim 1, further configured to operate as a pseudo primary care physician or digital longitudinal caregiver via an AI Caregiver Agent, designed to maintain a persistent and evolving knowledge base of the patient's health history and perform personalized healthcare management.

13. The platform of claim 1, further comprising one or more AI Agents configured to coordinate between parties, prescribers, and pharmacies to monitor prescription dosage schedules, send timely refill reminders, check for non-adherence patterns, and engage in real-time conversations with patients to assess tolerance and side effects.

14. The platform of claim 1, wherein one or more dedicated AI agents provide near real-time pain management assessment capabilities leveraging established pain assessment methodologies, gauge pain intensity, monitor progression, and correlate symptoms with treatment plans.

15. The platform of claim 1, wherein the AI agentic framework provides patients with context-based education resources approved by the health institution, including individualized information such as dietary restrictions, medications, and procedure preparation/recovery.

16. The platform of claim 1, wherein the AI agentic framework enables intelligent collaboration among patients, physicians, and nurses through an AI Triage Assist Agent, which ingests and evaluates information to assist triage conversations and facilitate access to clinical data and insights.

17. A method for enhancing patient personalized care outcomes and healthcare facility efficiencies using a conversational artificial intelligence (AI) platform, the method comprising:
receiving patient input through a multi-modal communication channel;
processing said patient input using natural language processing (NLP) techniques and Large Language Models (LLMs);
activating one or more specialized AI agents from an AI agentic framework based on the processed input, wherein each AI agent is configured for a distinct task;
collecting situational data and monitoring vital health data, including integration with wearable and non-wearable devices;
integrating collected data with patient historical Electronic Medical Record (EMR) data;
performing near real-time analysis and generating personalized recommendations or task executions using said AI agents and LLMs;
engaging in conversational interactions to respond to patient queries and autonomously initiating appropriate actions based on those interactions;
streamlining administrative tasks and facility-level workflows using said AI agents; and
delivering personalized care interventions and communications to the patient.

18. The method of claim 17, further comprising dynamically chaining or interconnecting two or more distinct AI agents from the AI agentic framework in real-time to collectively execute complex problems for a facility, physicians, or nursing staff.

19. The method of claim 17, further comprising enabling clinicians, administrators, or IT staff to design, modify, and deploy custom AI-driven workflows without writing code, through a graphical workflow design interface.

20. The method of claim 17, wherein the patient input is received through integration with AI-NLP front ends including Alexa, Google Home, and Siri.
